(19) United States
(12) Patent Application Publication
(10) Pub. No.: US 2024/0419496 A1
(43) Pub. Date: Dec. 19, 2024

(54) DISTRIBUTED SYSTEM OF COMPUTER ARCHITECTURES

(71) Applicant: Yale University, New Haven, CT (US)

(72) Inventors: Abhishek Bhattacharjee, New Haven, CT (US); Karthik Sriram, New Haven, CT (US); Raghavendra Pradyumna Pothukuchi, New Haven, CT (US); Rajit Manohar, New Haven, CT (US); Anurag Khandelwal, New Haven, CT (US)

(21) Appl. No.: 18/744,194

(22) Filed: Jun. 14, 2024

Related U.S. Application Data
(60) Provisional application No. 63/508,760, filed on Jun. 16, 2023.

(51) Int. Cl. G06F 9/50; G06F 1/3296

(52) U.S. Cl. CPC G06F 9/5027; G06F 1/3296

(57) ABSTRACT

Provided herein are methods for communicating data between nodes in a computer system and distributed systems of computer architectures. The methods include generating a hash based upon a data set, communicating the hash to one or more nodes, comparing the communicated hash to stored hashes at the one or more nodes, and communicating the data set when matching hashes are detected. The system includes two or more processing elements configured to communicate data according to the methods above.

### CROSS-REFERENCE TO RELATED APPLICATIONS

The present application claims priority under 35 U.S.C. § 119(e) to U.S. Provisional Patent Application No. 63/508,760, filed Jun. 16, 2023, which application is incorporated herein by reference in its entirety.

### STATEMENT REGARDING FEDERALLY SPONSORED RESEARCH OR DEVELOPMENT

This invention was made with government support under grant numbers 2118851, 2127309, and 2112562 awarded by NSF. The government has certain rights in the invention.

### BACKGROUND OF THE INVENTION

In certain instances, it is desirable to have multiple ultra-low power nodes communicate data with one another, such as, for example, communication between implants to converge on a joint plan of actuation. However, there are two main challenges with implant-to-implant communication. The first is that all the communication and processing on the implants has to be performed under a tight power budget to avoid damaging surrounding tissue. The second is that all data needed to be exchanged within a small window of time to ensure that appropriate action is taken to quickly enough to be effective. Both these goals are challenging to meet because of the sheer volume of data that has to be communicated. This same problem exists in many other settings with ultra-low power nodes or sensor networks that need to communicate within a window of time to jointly resolve a problem.

Accordingly, there is a need in the art for articles and methods that improve on existing articles and methods for communicating between low power nodes. The present invention addresses this need.

### SUMMARY OF THE INVENTION

In one aspect, provided herein is a method for communicating data between nodes in a computer system, the method including generating a hash based upon a data set; communicating the hash to one or more nodes; comparing the communicated hash to stored hashes at the one or more nodes; and communicating the data set when matching hashes are detected.

In some embodiments, detecting matching hashes includes determining that the underlying data sets are likely to be correlated. In some embodiments, determining that the underlying data sets are likely to be correlated includes applying a suitable similarity measure. In some embodiments, the similarity measure includes Euclidean distance, cross-correlation (XCOR), dynamic time warping (DTW), Earth Mover's Distance (EMD), or a combination thereof.

In some embodiments, at least one of the nodes locally generates and stores one or more of the hashes. In some embodiments, the at least one node locally generates and stores the one or more hashed based upon locally collected data. In some embodiments, the at least one node includes an originating node and a receiving node. In some embodiments, the originating node communicates the locally generated hash to the receiving node. In some embodiments, the receiving node compares the locally generated hash communicated from the originating node to a stored hash locally generated at the receiving node. In some embodiments, the receiving node responds to the originating node when there is a match between the locally generated hash communicated from the originating node and the stored hash locally generated at the receiving node. In some embodiments, the originating node communicates the locally collected data only when the receiving node responds.

In another aspect, provided herein is a distributed system of computer architectures including two or more processing elements; wherein the processing elements are configured to communicate data according to any of the methods disclosed herein. In some embodiments, the processing elements are connected through a wireless network, a wired network, wires on a chip, or a combination thereof. In some embodiments, the processing elements include application-specific integrated circuits (ASICs). In some embodiments, the ASICs are configured to realize a plurality of hashes in low power. In some embodiments, each of the processing elements is built in an independent clock domain. In some embodiments, the distributed system comprises a brain-computer interface (BCI) architecture for multi-site brain interfacing in real time. In some embodiments, the system is resource-constrained. In some embodiments, the system enables at least one distributed application selected from internal closed-loop applications, external closed-loop applications, and interactive human-in-the-loop applications. In some embodiments, the processing elements are low latency and low power.

### BRIEF DESCRIPTION OF THE DRAWINGS

FIGS. 1A-C show schematics illustrating an overview of BCI applications supported by SCALO. (A) Seizure propagation analysis. (B) Decoding movement intent with three different approaches. (C) Spike sorting to separate the combined electrode activity.

FIGS. 2A-B show schematics illustrating that the SCALO BCI is a distributed network of nodes implanted in multiple brain sites. The nodes communicate wirelessly with each other and the environment. Each SCALO node has sensors, radios, analog/digital conversion, processing fabric, and storage; the processing fabric contains hardware accelerators and configurable switches to create different pipelines. (A) SCALO overview. (B) The processor fabric in each of SCALO's nodes.

FIGS. 3A-C show schematics illustrating high-level overview of the BCI applications supported for online distributed processing in SCALO. (A) Seizure propagation analysis. (B) Decoding movement intent and stimulating as a response. (C) Spike sorting.

FIG. 4 shows a schematic illustrating programming and Interacting with SCALO.

FIG. 5 shows a schematic illustrating seizure detection and propagation on SCALO. The colors of the PEs are matched with the high-level tasks from FIG. 3A.

FIGS. 6A-C show schematics illustrating movement intent on SCALO. (A) Algorithm A. (B) Algorithm B. (C) Algorithm C.

FIG. 7 shows a schematic illustrating spike sorting on SCALO.

FIGS. 8A-C show graphs illustrating experimental quantification of SCALO's benefits. (A) Maximum aggregate throughput of SCALO and alternative BCI architectures for 11 nodes. (B) Maximum aggregate throughput of signal similarity methods. (C) Maximum aggregate throughput of movement intent applications.

FIGS. 9A-B show graphs illustrating application level metrics on SCALO. (A) Weighted throughput of seizure propagation tasks. (B) Movement intents per second.

FIG. 10 shows a graph illustrating interactive query throughput with 11 nodes.

FIG. 11 shows a graph illustrating hash errors.

FIG. 12 shows a graph illustrating network errors.

FIG. 13 shows a graph illustrating impact of radio.

FIG. 14 shows a graph illustrating hash flexibility.

FIGS. 15A-B show graphs illustrating maximum delay in detecting seizure propagation due to hash errors, averaged over all seizures. Shaded regions show the full range of observations. (A) Encoding errors. (B) Network errors.

### DETAILED DESCRIPTION OF THE INVENTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably +5%, even more preferably 10%, and still more preferably +0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein are methods for communicating data between nodes in a computer system. In some embodiments, the method includes establishing a hash-based communication system for filtering communication between the nodes. In some embodiments, the hash-based communication system includes generating a hash based upon a data set, communicating the hash to one or more nodes, comparing the communicated hash to stored hashes at the one or more nodes, and communicating the data set when matching hashes are detected and/or the underlying data sets are likely to be correlated. The comparing of the hashes to determine whether the underlying data sets are likely to be correlated includes any suitable similarity measure, such as, but not limited to, Euclidean distance, cross-correlation (XCOR), dynamic time warping (DTW), Earth Mover's Distance (EMD), any other measure for correlation, or a combination thereof.

In some embodiments, the hash-based communication system includes locality sensitive hashing (LSH), where hashes are generated and/or stored locally at one or more nodes. For example, one or more nodes may each individually collect and/or receive data locally, generate hashes using the locally collected and/or received data, and/or store the data/hashes locally. In such embodiments, each of the one or more nodes may individually act as an originating node that communicates one or more of the locally generated hashes to at least one of the other nodes acting as a receiving node. Each of the receiving nodes compares the one or more hashes communicated from the originating node to locally stored hashes and, when there is a match, responds to the originating node. Upon receiving a response from one or more of the receiving nodes, the originating node communicates the full data set used to generate the matching hash.

The hashes generated according to one or more of the embodiments disclosed herein are significantly smaller than the underlying data, and are generated quickly and accurately. For example, in some embodiments, the hashes are 100 times smaller than the underlying data. Additionally, the hash functions according to one or more of the embodiments disclosed herein are designed such that their output collides when their input (the collected/received data) are likely to be similar, resulting in transmission of the full data set only when the data sets are likely to be correlated (i.e., the hashes collide). Accordingly, the local computation and subsequent communication of hashes reduces the volume of data transmission and system power by orders of magnitude, as compared to the communication of the underlying data itself. The methods also reduce real-time latency in such systems.

As will be appreciated by those skilled in the art, the methods disclosed herein may be applied in various different systems. In some embodiments, the system includes a resource-constrained computer system, such as, but not limited to, any distributed system where nodes are communicating large amounts of information while operating at stringent power constraints and meeting real-time latency constraints. One such system includes brain-computer interfaces (BCIs), such as those described in more detail in the Examples below. Although described primarily with respect to BCIs in the Example below, the disclosure is not so limited and may be applied to other systems including, but not limited to, autonomous vehicles, nanosatellites, and other devices that must continuously sense and communicate in mission-critical settings.

In some embodiments, the method also includes decomposing classifiers, such as support vector machines (SVMs) and/or neural networks (NNs). Decomposing the classifiers reduces the dimension of data being communicated. Additionally or alternatively, in some embodiments, as opposed to the conventional approach of applying a classifier to all underlying data from all nodes, each of nodes calculates a partial classifier output on its own locally collected data. All outputs are then aggregated on a node to calculate the final result. Since local classifier outputs are 100× smaller than the raw inputs; communicating the former rather than the latter reduces network usage significantly.

Additionally or alternatively, in some embodiments, the method includes centralizing the matrix inversion operation used in a Kalman filter (e.g., used to decode movement intent). The Kalman filter generates large matrices as intermediate products from lower-dimensional electrode features, and inverts one such matrix. Distributing (and communicating) large matrices over a wireless (and serialized) network may exceed the response time goals for a specific application. Therefore, in such embodiments, the data is directly sent from all sites to a single node which computes the filter output, including the intermediate inversion step.

Also provided herein are articles configured to communicate data according to the methods disclosed herein. In some embodiments, the articles include processing elements (PEs), such as, but not limited to, those described in the Example below. In some embodiments, the articles form a system of computer architectures, such as, but not limited to, a distributed system of nodes connected in any suitable manner. For example, the nodes may be connected via a wireless network, a wired network (e.g., LAN), wires on a chip, any other suitable manner of connection, or a combination thereof. In some embodiments, the articles include a distributed system of ultra-low-power accelerator rich power-constrained computer architectures with two-step hash-based communication according to the methods disclosed herein.

In some embodiments, the articles include a family of hardware units that can be configured in a variety of ways to operate as appropriate hashes for different similarity measures (e.g., dynamic time warping, earth mover's distance, and cross-correlation). In some embodiments, and in contrast to existing approaches where one hardware hash is built for each of these separately, the articles include application-specific integrated circuits (ASICs) that can be configured to realize a plurality of hashes in low power. In some embodiments, the hardware units, such as sub-hash ASICs, are built in separate clock domains at just the clock rates that they need to run in order to provide the overall hash for the system. In such embodiments, the hashes may be modularly upgraded.

In some embodiments, the articles form a BCI architecture for multi-site brain interfacing in real time. In such embodiments, the articles include a distributed system of wirelessly networked implants. In some embodiments, each implant includes a HALO processor augmented with storage and compute to support distributed BCI applications. In some embodiments, the system includes an integer linear programming (ILP)-based scheduler that maps applications to the accelerators and creates network/storage schedules to feed the hardware accelerators. In some embodiments, the system includes a programming interface that is easily plugged into widely-used signal processing frameworks (e.g., TrillDSP, XStream, and MAT-LAB). In some embodiments, the articles support existing single-implant applications (HALO), and also enable three new classes of distributed applications.

The first class includes internal closed-loop applications that operate (e.g., modulate brain activity) without communicating with external systems. When applied to BCI, these applications monitor multiple brain sites, and when necessary, respond autonomously with electrical stimulation. Examples include detection and treatment of epileptic seizure spread, essential tremor, and Parkinson's disease.

The second class includes external closed-loop applications where the system communicates with other external systems (e.g., BCIs communicating with systems external to the brain and BCI). Examples include neural prostheses for speech and brain-controlled screen control devices.

The third class includes interactive human-in-the-loop applications, where operators (e.g., clinicians) query the system (e.g., BCI) for data or dynamically adjust processing/stimulation parameters. This is useful for many applications, such as, but not limited to, validating BCI detection of seizures, personalizing stimulation algorithms to individuals, or debugging BCI operation.

By tightly codesigning compute with storage, networking, scheduling, and application layers, the articles and methods disclosed herein achieve ultra power-efficient operation. For example, in some embodiments, the communication between nodes in the distributed system is reduced by: (1) building locality-sensitive hash measures to filter candidates for expensive signal similarity analysis across nodes; (2) reducing data dimensionality by hierarchically splitting computations in classifiers and neural networks; and, unusually, (3) by centralizing rather than distributing key computations when appropriate (e.g., like matrix inversion).

In some embodiments, the articles include hardware accelerators or processing elements (PEs) to support (1)-(3) above with low latency and power. The PEs may be reconfigurable to realize many applications and/or composed in a GALS (Globally Asynchronous Locally Synchronous) architecture. Additionally or alternatively, in some embodiments, each PE is realized in an independent clock domain, which allows it to be tuned for the minimal power to sustain a given application-level processing rate. In some embodiments, the articles include per-node non-volatile memory (NVM) to store prior data and hash data. The storage layout may be optimized for PE access patterns.

In some embodiments, the system includes per-node radios that support an ultra-wideband (UWB) wireless network. In some embodiments, the PEs directly access the network and storage, avoiding the bottlenecks that traditional accelerator-based systems (including ultra-low-power coarse-grained reconfigurable arrays or CGRAs) suffer in relying on CPUs to orchestrate data movement.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

### Example 1

### Abstract

SCALO is the first distributed brain-computer interface (BCI) consisting of multiple wireless-networked implants placed on different brain regions. SCALO unlocks new treatment options for debilitating neurological disorders and new research into brain-wide network behavior. Achieving the fast and low-power communication necessary for real-time processing has historically restricted BCIs to single brain sites. SCALO also adheres to tight power constraints, but enables fast distributed processing. Central to SCALO's efficiency is its realization as a full stack distributed system of brain implants with accelerator-rich compute. SCALO balances modular system layering with aggressive cross-layer hardware-software co-design to integrate compute, networking, and storage. The result is a lesson in designing energy-efficient networked distributed systems with hardware accelerators from the ground up.

### 1. INTRODUCTION

Brain-computer interfaces (BCIs) connect biological neurons in the brain with computers and machines. BCIs are advancing our understanding of the brain, helping treat neurological/neuropsychiatric disorders, and helping restore lost sensorimotor function. BCIs are also enabling novel human-machine interactions with new applications in industrial robotics and personal entertainment.

BCIs sense and/or stimulate the brain's neural activity using either wearable surface electrodes, or through surgically implanted surface and depth electrodes. BCIs have historically simply relayed the neural activity picked up by electrode sensors to computers that process or “decode” that neural activity. But, emerging neural applications increasingly benefit from BCIs that also include processing capabilities. Such BCIs enable continuous and autonomous operation without tethering.

This Example focuses on the design of processors for surgically implanted BCIs that are at the cutting edge of neural engineering. Although they pose surgical risks, implanted BCIs collect far higher fidelity neural signals than wearable BCIs. Consequently, implantable BCIs are used in state-of-the-art research applications and have been clinically approved to treat epilepsy and Parkinson's disease, show promise (via clinical trials) in restoring movement to paralyzed individuals, offer a path to partially restoring vision to visually-impaired individuals, and more.

Implantable BCI processors are challenging to design. They are limited to only a few milliwatts of power as overheating the brain by just >1° C. risks damaging cellular tissue. At the same time, implantable BCIs are expected to process exponentially growing volumes of neuronal data within milliseconds. Most modern BCIs achieve low power by specializing to a single task and by sacrificing neural processing data rates. Neither option is ideal. BCIs should instead be flexible, so that algorithms on board can be personalized to individuals and so that many new and existing algorithms can be supported. And, BCIs should process higher data rates to infer more about the brain. To achieve these goals, the present inventors have proposed HALO, an accelerator-rich processor that achieves low power at neural data rates orders of magnitude higher than prior work (46 Mbps), but also achieves flexibility via programmable inter-accelerator data flow.

While HALO successfully balances power, data rate, and flexibility, it interfaces with only a single brain site, whereas future BCIs will consist of distributed implants that interface with multiple brain sites. Applications that process neural data from multiple brain sites over multiple timescales are becoming common as neuroscience research is increasingly showing that the brain's functions (and disorders) are based on temporally-varying physical and functional connectivity among brain regions. Assessing brain connectivity requires placing communicating implants in different brain regions, with storage that enables multi-timescale analysis. Unfortunately, no existing BCIs integrate adequate storage for such long-scale analysis. Even worse, communication is problematic. Because wired networks impose surgical risk and potential infection, wireless networking is desirable. Unfortunately, however, wireless networking offers lower data rates (10× lower than compute) under milliwatts of power.

These challenges are addressed herein by proposing and building SCALO, the first BCI architecture for multi-site brain interfacing in real time. SCALO is a distributed system of wirelessly networked implants. Each implant has a HALO processor augmented with storage and compute to support distributed BCI applications. SCALO includes an integer linear programming (ILP)-based scheduler that optimally maps applications to the accelerators and creates network/storage schedules to feed the hardware accelerators. SCALO has a programming interface that is easily plugged into widely-used signal processing frameworks like TrillDSP (Milos Nikolic, Badrish Chandramouli, and Jonathan Goldstein. 2017. Enabling Signal Processing over Data Streams (SIGMOD '17). Association for Computing Machinery, New York, NY, USA, 95-108. https://doi.org/10.1145/3035918. 3035935), XStream (Lewis Girod, Yuan Mei, Ryan Newton, Stanislav Rost, Arvind Thiagarajan, Hari Balakrishnan, and Samuel Madden. 2008. XStream: a Signal-Oriented Data Stream Management System. In 2008 IEEE 24th International Conference on Data Engineering. 1180-1189. https://doi.org/10.1109/ICDE.2008.4497527), and MAT-LAB (Starting Matlab. 2012. Matlab. The MathWorks, Natick, MA (2012)). SCALO continues to support HALO's single-implant applications (Ioannis Karageorgos, Karthik Sriram, Ján Vesely’, Michael Wu, Marc Powell, David Borton, Rajit Manohar, and Abhishek Bhattacharjee. 2020. Hardware-software co-design for brain-computer interfaces. In 2020 ACM/IEEE 47th Annual International Symposium on Computer Architecture (ISCA). IEEE, 391-404. https://doi.org/10.1109/ISCA45697.2020.00041), but also enables, for the first time, three new classes of distributed applications.

The first class consists of internal closed-loop applications that modulate brain activity without communicating with systems external to the BCI. These applications monitor multiple brain sites, and when necessary, respond autonomously with electrical stimulation. Examples include detection and treatment of epileptic seizure spread, essential tremor, and Parkinson's disease.

The second class consists of external closed-loop applications where BCIs communicate with systems external to the brain and BCI. Examples include neural prostheses for speech and brain-controlled screen control devices.

The third class consists of interactive human-in-the-loop applications where clinicians query the BCI for data or dynamically adjust processing/stimulation parameters. This is useful to validate BCI detection of seizures, personalize stimulation algorithms to individuals, or debug BCI operation.

SCALO achieves ultra power-efficient operation by tightly codesigning compute with storage, networking, scheduling, and application layers. Knowledge of neural decoding methods was used to reduce communication between implants comprising the distributed BCI by: (1) building locality-sensitive hash measures to filter candidates for expensive signal similarity analysis across implants; (2) reducing data dimensionality by hierarchically splitting computations in classifiers and neural networks; and, unusually, (3) by centralizing rather than distributing key computations when appropriate (e.g., like matrix inversion in our applications).

SCALO consists of hardware accelerators or processing elements (PEs) to support (1)-(3) above with low latency and power. The PEs were built so that they can be reconfigured to realize many applications, and composed in a GALS (Globally Asynchronous Locally Synchronous) architecture. By realizing each PE in its independent clock domain, it is allowed to be tuned for the minimal power to sustain a given application-level processing rate. Per-implant non-volatile memory (NVM) was used to store prior signals and hash data. The storage layout is optimized for PE access patterns.

SCALO also includes per-implant radios that support an ultra-wideband (UWB) wireless network. The PEs were built to directly access the network and storage, avoiding the bottlenecks that traditional accelerator-based systems (including ultra-low-power coarse-grained reconfigurable arrays or CGRAs) suffer in relying on CPUs to orchestrate data movement.

SCALO's components are predictable in latency and power, facilitating optimal compute/network scheduling with an ILP. For PEs whose output data generation rates are based on input patterns (e.g., data compression), the present ILP uses worst-case bounds.

SCALO was evaluated with a physical synthesis flow in a 28 nm CMOS process coupled with network and storage models. The evaluations are supported by prior partial chip tape-outs of HALO in a 12 nm CMOS process. SCALO achieves an aggregate neural interfacing data rate of 506 Mbps using 11 implants to assess and arrest seizure propagation within 10 ms of seizure onset; 188 Mbps using 4 implants to relay intended movements to external prostheses within 50 ms and restore sensorimotor function; and sorts 12,250 spikes per second per site with a latency of 2.5 ms. All applications expend less than 15 mW per implant. When used for interactive querying, SCALO supports 9 queries per second over 7 MB of data over 11 implants. Overall, the contributions include:
(1) A full-stack accelerator-rich distributed BCI, with unusually tight integration of compute with network and storage.
(2) The design of an optimal ILP-scheduler for mapping applications across distributed accelerators and network, enabled by a deterministic compute, network and storage design.
(3) An interface facilitating easy integration into existing data and signal processing platforms.

These technical contributions, in turn, translate into advances in neural decoding and computer systems design:
(1) Neural Decoding: The first distributed wireless BCI processing architecture for decoding, analysis, and electrical stimulation of brain-wide networks. SCALO offers the first on-device support for seizure propagation and movement intent analysis on multiple brain sites. SCALO includes configurable on-device locality sensitive hashing for fast signal similarity analysis.
(2) Computer Systems: An experiment in the design of an end-to-end distributed system of accelerators from the application layer to physical synthesis. The evaluation shows 10-385× higher processing rates over prior work at 15 mW per implant.

### 2. BACKGROUND

### 2.1 Components of a BCI

BCI applications include signal measurement, feature extraction, classification/decision-making, and when applicable, neural feedback/stimulation. BCIs include hardware components mirroring each of these four stages.

Signal measurement is performed by electrodes that read the electrical activity of neurons and analog-to-digital converters (ADCs) that digitize these signals. Arrays of 96-256 electrodes or depth probes of 1-4 electrodes are widely used. BCI ADCs typically sample at 5-50 KHz per electrode with 8-16 bit resolution.

Feature extraction and classification/decision-making are performed on the digitized data. These portions of the neural pipeline were historically undertaken by external servers, but on-BCI computation is becoming increasingly important. When neural feedback is needed, the electrodes are repurposed (after digital-to-analog conversion) to electrically stimulate the brain. Electrical stimulation can, for example, mitigate seizure symptoms.

Traditional BCI communication with external server/prostheses relied on wires routed through a port embedded in the skull. But, wiring restricts the individual's movement, hinders convenience, and is susceptible to infections and cerebrospinal fluid leaks. Wireless radios avoid these issues and are consequently being used more widely.

Some BCIs use batteries that are implanted and single-use or externally removable. Recent BCIs are using implanted rechargeable batteries with inductive power transfer.

Taken together, all these components are packaged in hermetically-fused silica or titanium capsules. While safe power limits depend on implantation location and depth, 15 mW per implant is used herein as a conservative limit.

### 2.2 BCI Applications & Kernels

The space of BCI applications is rapidly growing. Some require neural data from only a single brain region (e.g., spike sorting) while many others (e.g., epileptic seizure propagation and movement intent decoding) require neural data from multiple brain regions. Target herein are three classes of distributed BCI applications that operate in autonomous closed loops. From each class, a representative application is studied. Additionally, spike sorting, a kernel that is often used to pre-preprocess neural data before subsequent application pipelines, is also studied.

Internal closed-loop applications: Nearly 25 million individuals worldwide suffer from drug-resistant epilepsy and experience seizures that last tens of minutes to hours per day. BCI-led closed-loop therapy can help these individuals immensely. SCALO supports epileptic seizure propagation calculations on device, which many recent studies show as being desirable. Seizure propagation applications correlate neural signals from brain regions where seizures originate to current and historical signals from other brain sites. Correlations help identify the network dysfunction that underlies seizure spread, which in turn unlocks targeted treatment options.

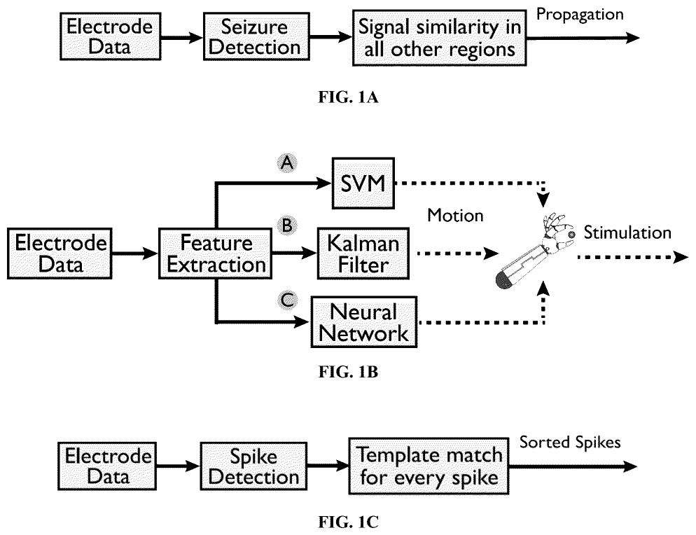

FIG. 1A illustrates seizure propagation analysis. First, seizures are detected “locally” in each brain site. This is done with band-pass filtering and/or the fast Fourier transform (FFT), which generate features from contiguous time windows of neural data, and then using classifiers like support vector machines (SVMs).

When a seizure is detected at a brain site, its neural data is correlated with recent and past neural signals from other brain sites. Many measures are used to determine correlation, including dynamic time warping (DTW), Euclidean distance, cross-correlation, and Earth Mover's Distance (EMD). Once correlated brain regions are identified and seizure spread is forecast, brain regions anticipating seizure spread are electrically stimulated to mitigate the spread.

Treatment effectiveness depends on accurate but also timely seizure forecasting. In this Example, a challenging 10 ms target from local seizure detection to seizure forecasting and electrical stimulation was set.

External closed-loop applications: These applications help individuals control assistive devices external to BCIs like artificial limbs, cursors on computer screens, or prostheses that translate brain signals corresponding to intended speech into text on computer screens. Three neural processing algorithms representative of this category of applications were selected and are illustrated in FIG. 1B.

Pipeline A classifies neural activity into one of a preset number of limb movements like finger pointing, arm stretch, and more. The features are extracted using FFT and filters, and used by a classifier to identify movement. Linear SVMs are most commonly used for classification. More complex deep neural networks (DNNs) have been shown to outperform SVMs and are promising. For now, SCALO supports linear SVMs and shallow networks as they require less training data than DNNs, have more intuitive parameter tuning, and are more interpretable. However, it is believed that SCALO can also support DNNs.

Unlike pipeline A (which identifies complex movements as a whole), pipelines B and C decode the position and velocity of arm/finger movements or cursor movements on screen. Pipelines B and C calculate spike band power in neural signals by taking the mean value of all neural signals in a time window (typically 50 ms). Pipelines may use a variant of Kalman Filter (B) or a shallow neural network (C) to decode movement intent.

Decoded intended movement is relayed to computer screens, artificial limbs, or even paralyzed limbs implanted with electrodes. When the individual has also lost sensory function, the “feeling” of movement is emulated by relaying the impact of the movement back to the individual's BCI. The BCI then electrically stimulates relevant brain sites to emulate sensory function. The entire movement decoding loop must complete within 50 ms to effectively restore sensorimotor control.

Human-in-the-loop applications: Researchers or clinicians wish to interactively query BCI devices. They may retrieve important neural data, configure device parameters for personalization, or verify correct operation. Low query latency is not just desirable, but often necessary. For example, a clinician may need to retrieve neural data and manually confirm that the BCI correctly detected a seizure. Or, a clinician may plan to test the effectiveness of a new electrical stimulation protocol for treatment. Faster device querying measurably improves BCI utility in such cases. It is important, however, that interactive querying does not disrupt the other BCI applications that are continuously running.

Spike sorting kernel: Each electrode usually measures the combined electrical activity of a cluster of spatially-adjacent neurons. This combined activity is also influenced by sensor time lag, signal attenuation, and sensor drift. The goal of spike sorting is to separate combined neural activity into per-neuron waveforms. Unlike the applications discussed thus far, spike sorting is entirely local to each brain site. But, it is a widely used first step for important BCI applications that rely on neuron-level analysis. In fact, spike sorting would also benefit other applications like movement intent decoding if it could be made faster (today, the prohibitive cost of spike sorting prompts usage of approximated sorting). SCALO offers power-efficient spike-sorting within a few milliseconds to fully unlock its potential.

FIG. 1C shows a typical spike sorting pipeline. Spike waveforms are detected from electrode signals. They are then matched with templates corresponding to each neuron. Such templates may be obtained offline from prior recordings or generated online with clustering. Spike waveforms are matched with templates using some of the same compute-intensive correlation measures from seizure propagation pipelines; e.g., DTW and EMD.

### 2.3 BCI Design Challenges

BCIs cannot exceed 15 mW and have tight response times (10 ms for seizure propagation, 50 ms for movement decoding, a few 100 ms for interactive querying, and a few milliseconds for spike sorting). Distributed processing is challenging because inter-implant communication radios have low data rates, and do not use multiple frequencies (to save power), requiring serial network access.

Overspecializing hardware to achieve low power is undesirable. Neural signaling differs across brain regions and across subjects. Signaling even evolves over time, and as a consequence of the brain's response to the implant. No single processing algorithm and parameters is optimal for the application pipelines in Section 2.2. Instead, these pipelines must be customized to the implant site, the individual, and must be regularly re-calibrated.

Distributed BCIs heighten this tension in power and flexibility. The few distributed multi-site BCIs that have been built to date use multiple sensor implants that offload processing to external computers, but do not support on-BCI processing. This restricts their scope and timeliness.

SCALO is the first distributed multi-site BCI to offer on-BCI processing. One may initially expect thermal coupling between the implants in SCALO to restrict per-implant power budgets below the 15 mW target of single-site BCIs like HALO. As detailed in Section 5, however, the brain's cerebrospinal fluid and blood flow dissipate heat effectively on the cortex, making thermal coupling negligible even with relatively short inter-implant spacing.

### 2.4 Locality-Sensitive Hashing

While inter-implant thermal coupling is less of a concern, inter-implant communication latency becomes the barrier to the design of a wireless-networked multi-site BCI. In response, the methods discussed herein lean on locality sensitive hashing (LSH), a technique used for fast signal matching. LSH offers a way to filter inter-implant communication to only those neural signals most likely to be correlated (as determined by similarity measures like DTW, EMD, etc.).

SCALO's design is based on LSH approaches for DTW and EMD. LSH approaches for DTW first create sketches of neural signals by calculating the dot product of sliding windows in the signal with a random vector. The sketch of a window is 1 if the dot product is positive and 0 otherwise. Then, the occurrences of all n-grams formed by n consecutive sketches are counted. The n-grams and their counts are used by a randomized weighted min-hash step to produce the hash. The LSH for EMD calculates the dot product of the entire signal with a random vector, and then computes a linear function of the dot product's square root.

### 3. THE SCALO ARCHITECTURE

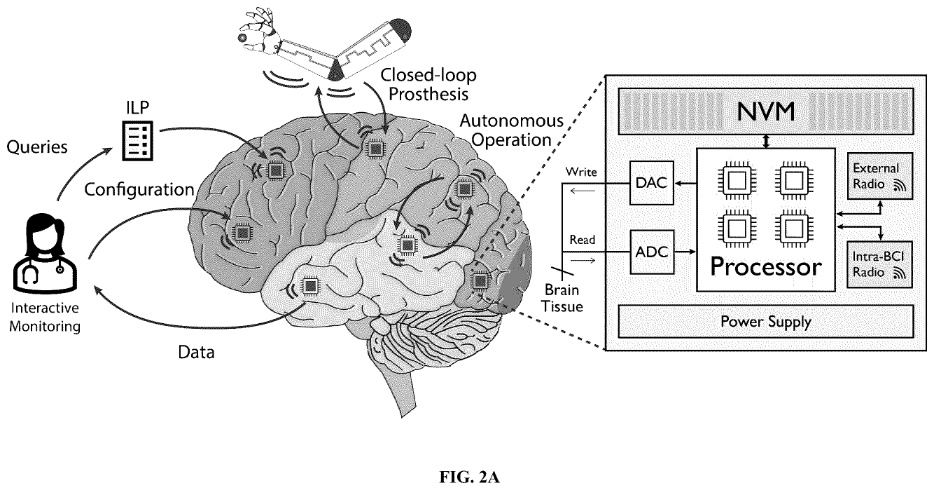

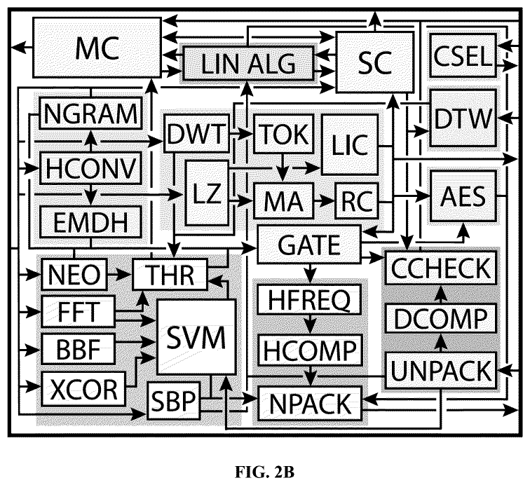

FIGS. 2A-B show SCALO and its implants (or nodes). Each SCALO node contains 16-bit ADCs/DACs, an accelerator/PE-rich reconfigurable processor, an NVM layer, a radio for inter-node (intra-BCI) communication and another radio for external communication, as well as a power supply. SCALO can run various applications and interactive queries expressed widely-used high-level languages. An ILP scheduler maps their operations onto the nodes optimally.

### 3.1 On-BCI Distributed Neural Pipelines

The first step was to convert the pipelines in FIGS. 1A-C into counterparts amenable for distributed processing. One enhancement is to enable the pipelines to use storage to assess correlations over multiple timescales, while another is to modify the pipeline to mitigate the inter-node communication bottleneck.

First, signal comparison was split into a fast hash check, and subsequent exact comparison. The hash check identifies neural data that is (in high probability) uncorrelated among brain regions, and hence unnecessary for inter-node exchange. Hashes are 100× smaller than signals, and can be quickly and accurately generated. They significantly filter compute and inter-node communication.

Second, classifiers like SVMs and neural networks (NNs) were decomposed to reduce the dimension of data being communicated. Instead of the conventional approach of applying a classifier to all neural data from all brain sites, each of SCALO's nodes calculates a partial classifier output on its own data. All outputs are aggregated on a node to calculate the final result. Local classifier outputs are 100× smaller than the raw inputs; communicating the former rather than the latter reduces network usage significantly. Decomposing linear SVMs is trivial and does not affect accuracy. NNs are similarly decomposed by distributing the rows of the weight matrices.

Third, the matrix inversion operation used in the Kalman filter was centralized. The Kalman filter generates large matrices as intermediate products from lower-dimensional electrode features, and inverts one such matrix. Distributing (and communicating) large matrices over our wireless (and serialized) network violates the response time goals set herein (Section 2.3). Therefore, the electrode features were directly sent from all sites to a single implant which computes the filter output, including the intermediate inversion step.

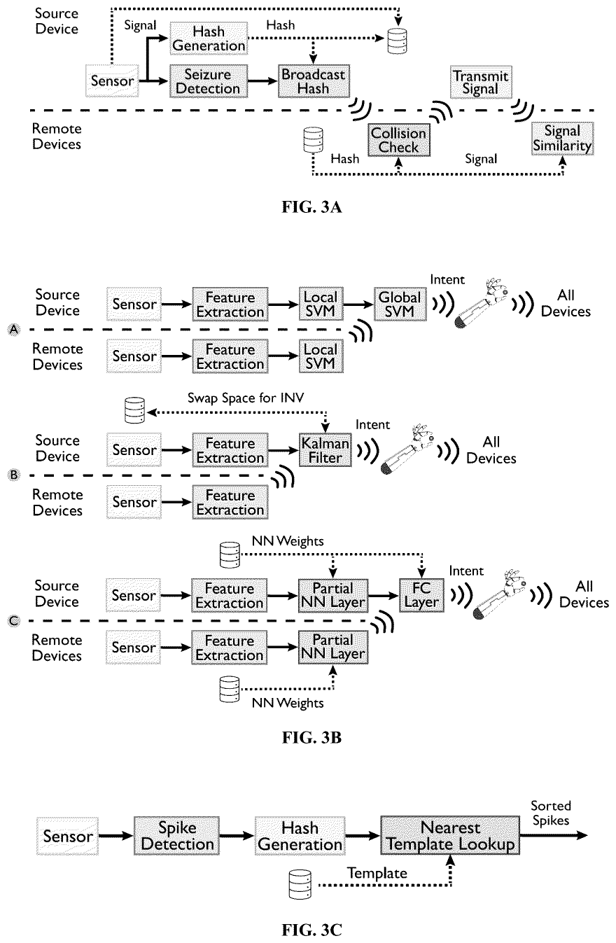

FIG. 3A shows a new distributed seizure propagation analysis according to the present Example. Each electrode's samples are collected in a sliding window (e.g., 120 samples) and then used to generate a hash. Hashes are stored in the NVM. When a SCALO node detects a seizure on one or more signal windows, it broadcasts the corresponding hashes to other nodes. Receiver nodes check if these hashes match with any of their recently stored local hashes and respond on a match. The original node broadcasts the full signal window corresponding to the matching hash. Receiver nodes confirm seizure propagation by exactly comparing their local signals with the received ones. Finally, electrical stimulation can be applied at all locations with a seizure spread. Importantly, local per-node seizure detection (omitted in FIG. 3A) continues unabated during this correlation step.

FIG. 3B shows a distributed movement decoding application according to the present Example. Algorithms A and C benefit from hierarchically decomposed SVMs and NNs. Each node computes a partial local output. A single node aggregates outputs and generates a final decision. In Algorithm B, each node extracts features locally and transmits them to a node running the Kalman filter to decode movement intent.

Finally, FIG. 3C shows an online spike sorting pipeline according to the present Example. Spike sorting benefits from hash-based signal processing and storage. Spikes from the incoming signals are detected, and encoded with hashes. These hashes are compared with the hashes of templates that are locally stored in each node to classify the spike waveforms. Since spike sorting is a precursor to advanced processing, the fast online version described herein can benefit many applications.

### 3.2 Flexible & Energy-Efficient Accelerators

SCALO's nodes are based on an augmented version of the present inventors' prior work, HALO. SCALO's PEs can be reused across applications, and have deterministic latency/power. Wide-reuse PEs minimize design and verification effort, and on-chip area. Deterministic latency/power enables simple and optimal application scheduling.

FIG. 2B shows the processor in each SCALO node. There are many PEs (functions described in the appendix) connected with programmable switches. The switches can be configured to realize various processing pipelines. Being a GALS design, it is easy to even configure PEs across multiple nodes in a pipeline. In addition to the PEs for single-site applications, new functionality has been incorporated to support distributed applications.

LSH support: Hash support has been built for four commonly-used signal similarity measures—Euclidean distance, cross-correlation (XCOR), DTW distance, and EMD.

Prior work has proposed an LSH specifically for DTW, but the present inventors discovered that by varying the LSH's parameters, it can also serve as a hash for Euclidean distance and cross-correlation. This discovery enables the design of a single LSH PE that can generate hashes for all three measures. To accommodate the LSH for EMD, a shared dot product with the LSH for DTW was identified (Section 2.4). In sum, three PEs were designed to support all LSHs: dot product computation (HCONV), n-gram count and weighted min-hash (NGRAM), and square root (EMDH).

A crucial aspect of the LSH PEs described herein is that the weighted min-hash calculation from prior work uses a variable-latency randomization step. To guarantee deterministic latency and power while preserving the LSH property, an alternative method is used herein.

When hashes are received by a node for matching, they are sent to the CCHECK PE that stores them in SRAM registers and sorts them in place. The PE reads local hashes up to a configurable past time (e.g., 100 ms) from the on-chip storage, and checks for matches with the received hashes using binary search.

Signal comparison: PEs are used for selecting the signals to be broadcast (CSEL) and for comparison (DTW, XCOR). The DTW PE uses a pipelined implementation of the standard DTW algorithm with a Sakoe-Chiba band parameter for faster computation. The same PE measures Euclidean distance by setting the band parameter to 1. Additionally, the XCOR PE from HALO is reused.

EMD is more computationally expensive than all other measures, but a fast version is sued for which the on-chip general-purpose microcontroller (described later) was sufficient. The PE may also be designed for the full EMD calculation.

Linear Algebra Computations: While HALO originally integrated an SVM PE, distributed applications require more complex linear algebra (e.g., matrix multiplication and inversion) for which linear algebra PEs (LIN ALG) were built. LIN ALG has PEs for matrix multiplication and addition with a constant matrix (MAD), matrix addition (ADD), subtraction (SUB), and inversion (INV). MAD can be configured to perform multiplication (MUL) only. All PEs use 16 KB registers with single-cycle access to store input matrices and constants. Larger entries can be read from the NVM.

Because SCALO does not support loops, applications with several MAD operations can be accelerated by either replicating MAD PEs or saving values to memory. For <10 MAD operations, the latency benefits of PE replication outweigh its hardware cost, and 10 MAD PEs are used in the LIN ALG cluster. Four MAD PEs are tiled into 4-way blocks to support large matrix operations found in the Kalman filter. Not all MAD PE units are tiled since the remaining operations in the Kalman filter use smaller matrices.

Rectified linear activation (ReLU) and normalization operations used in NNs are implemented by adding configurable parameters to the MAD and ADD units. When the ReLU parameter is set, the units suppress negative outputs by replacing them with 0. When normalization is set, the units read the mean and standard deviation as parameters and normalize the output. Matrix inversion is implemented in hardware using the Gauss-Jordan elimination method.

Networking Support: The intra-SCALO network carries hashes and signals/signal features. As network data rate is low, compression increases transmitted item count. However, because compression reduces redundancy, it also increases susceptibility to network errors. A balance is stricken based on the likelihood of errors.

Signals remain lengthy after compression (≈120-240 B) and can suffer errors for a given network bit error rate (BER). Measures like DTW are naturally resilient to errors for uncompressed signals, but lose accuracy using erroneous compressed signals. Signal features like those used to decode movement intent are lengthy and sensitive to errors when compressed. Compressing them is therefore avoided.

Hash comparison also fails quickly with erroneous hashes, but such errors are 100× less likely because hashes are short even before compression (1-2 B). Therefore, hashes are compressed. When hashes suffer an error, comparison can still proceed with subsequent hashes because brain signals at a site are temporally correlated. It is shown that it takes an unusually high BER to delay the application (e.g., seizure propagation) by 1 ms (Section 6.7).

HALO's PEs (i.e., LZ/LZMA) were originally built to transmit large volumes of data to external servers and are not suitable for low-latency hash compression. New PEs were developed to compress intra-SCALO communication. The HFREQ PE collects each node's hash values and sorts them by frequency of occurrence. The HCOMP PE applies multiple compression algorithms serially. It first encodes the hashes with dictionary coding, then uses run-length encoding of the dictionary indexes, and finally uses Elias-7 coding on the run-length counts. By customizing the compression strategy to the data, HCOMP achieves a compression ratio that is only 10% lower than that of LZ4/LZMA, but uses 7×less power.

Compressed data is sent to the NPACK PE, which adds check-sums before transmission. There are UNPACK and DCOMP PEs to decode and decompress packets respectively, on the receiving side.

Storage Control: Access to the on-chip NVM is managed by the SC PE. This PE has SRAM to buffer writes before they are sent to the NVM as 4 KB pages and during erase operations. The SRAM is also used to reorganize the data layout (Section 3.3) to speedup future reads from the NVM. Finally, SC uses registers to store metadata (e.g., the last written page) to speedup recent data retrieval.

Microcontroller: SCALO has a RISC-V microcontroller, MC, for several operations. It configures PEs into pipelines, and runs neural stimulation commands. The MC is also used for computations not supported by any PEs such as new algorithms, or infrequently run system operations such as clock synchronization (Section 3.6). The MC runs at a low frequency of 20 MHz and integrates 8 KB SRAM.

Optimal Power Tuning: Each of SCALO's PEs operates in its own clock domain, similar to the present inventors' prior work on HALO. However, HALO supported only one frequency per PE. This is not optimal for SCALO's applications, which sometimes operate only on a subset of electrode data. For example, seizure propagation requires exact comparison for only a few signals to remain under target response times. Running PEs at only one target frequency even when input data rates may be lower, wastes power.

Support was added for multiple frequencies per PE, and the lowest necessary was picked to sustain a target data rate, minimizing power. SCALO's PEs support a frequency fₘₐₓ^{PE}, high enough for the maximum data rate, and divide it to fₘₐₓ^{PE}/k, where k is user-programmable. Division is achieved using a simple state machine and counter that passes through every k clock pulses. The counter consumes only μWs. Multiple frequency rails were used to ensure the PE has the same latency despite variable number of inputs.

### 3.3 Per-Implant NVAM Storage

Each node integrates 128 GB NVM to store (in separate partitions) signals, hashes, and application data (e.g., weight matrices, spike templates). The MC uses a fourth NVM partition. Partition sizes are configurable. When full, the oldest partition data is overwritten.

We co-design the NVM data layout with PE access patterns to meet ms-scale response times. SCALO's ADCs and LSH PEs generate values sequentially by electrode. Stored as is, extracting a contiguous signal window of an electrode (used by most operations) requires reading from several discontinuous NVM locations. We reorganize neural data to store contiguous signals in chunks (with a configurable chunk size). This enables data retrieval with fast contiguous NVM reads. Our approach takes 5× longer for writes (1.75 ms), but is 10× faster for reads (0.035 ms). Data is written once but read multiple times, and writes are not on the critical path of execution, while reads are. These two factors make our approach more efficient. We reuse SC PE write buffers for this reorganization.

### 3.4 Networking

SCALO incorporates three networks. From the HALO work, the inter-PE circuit switched network and the wireless network to communicate with external devices up to 10 m were maintained. A new wireless network for intra-SCALO communication was added, using a custom protocol with TDMA. The ILP described herein generates a fixed network schedule across all the nodes (Section 3.5).

The intra-SCALO network packets have an 84-bit header, and a variable data size up to a maximum packet size of 256 bytes. The header and data have 32-bit CRC32 checksums. On an error, the receiver drops the packet if it contains hashes, but uses it if it has signals. This is because signal comparison measures like DTW are naturally resilient to a few errors. We evaluate the impact of allowing erroneous signal packets in Section 6.6.

### 3.5 Optimal System Scheduling

We use a software ILP-based scheduler to map tasks to PEs and generate storage and network schedules. The deterministic latency and power characteristics of our system components makes optimal software scheduling feasible. The scheduler takes as input the data flow graph of applications and queries, constraints like the response time, and priorities of application tasks/stages (e.g., seizure detection versus signal comparison). A higher priority for a task ensures that more electrodes signals are processed in it relative to the others when all signals cannot be processed in all tasks.

Each task is modeled as a flow, and the ILP maximizes the number of electrodes processed in each flow. It ensures that overall response time and power constraints are met. It is acceptable for two flows to share the same PE. In this case, the signals from each flow are interleaved and run at a single frequency, completing within the same time as if they were run independently. The hardware tags the signals from each flow to route them to the correct destinations.

### 3.6 System Maintenance

Clock synchronization: SCALO's distributed processing requires the clocks in each BCI node to be synchronized with a precision of a few μs. The clocks are based on pausable clock generators and clock control units that suffer only picoseconds of clock uncertainty, a scale much smaller than the present μs target. SCALO also operates at the temperature of the human body and does not experience clock drift due to temperature variance. Nevertheless, SCALO synchronizes clocks once a day using SNTP.

In SNTP, one SCALO node is designated as the server. All other nodes send messages to it to synchronize clocks. The clients send their previously synchronized clock times and current times, while the server sends its corresponding times. Clocks are adjusted based on the difference between these values. This process repeats until all the clocks are synchronized within the desired precision. During clock synchronization, the intra-SCALO network is unavailable for other operations, but tasks like seizure detection that do not need the network continue unimpeded.

Wireless Charging: Powering BCIs is an open problem, especially for distributed implants. It is assumed that the SCALO nodes are wirelessly powered, similar to prior demonstrations for distributed and centralized sensor implants (even though wired power delivery through a hub is also possible). When charging wirelessly, all pipelines are paused to avoid overheating. While charging frequency and duration varies by algorithm and battery technology, recent work has shown that it is possible to have 24-hour operation with 2 hours of charging.

### 3.7 Programming & Compilation

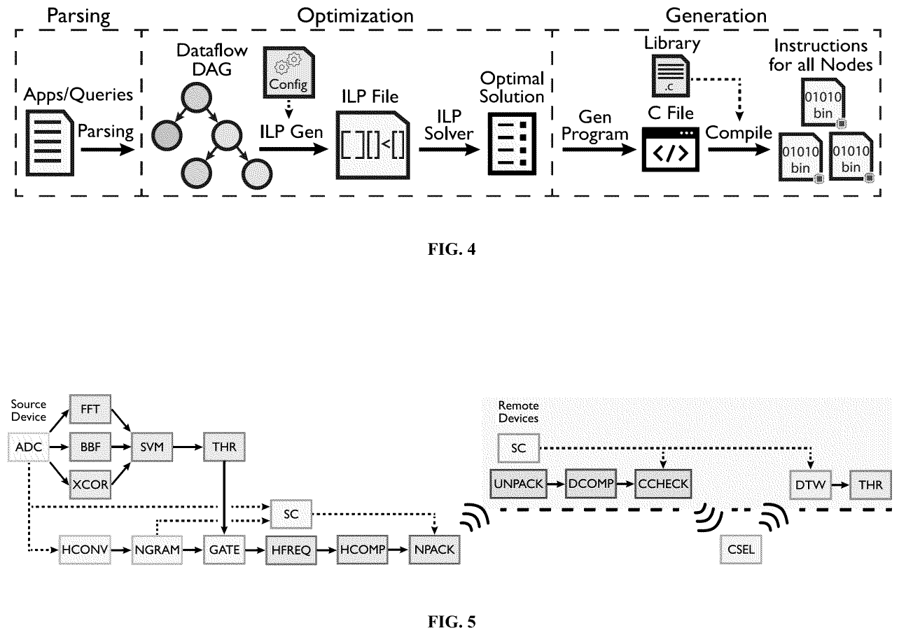

FIG. 4 shows how SCALO is programmed. Clinicians or neuroscientists create programs in popular high-level languages like MAT-LAB or TrillDSP to describe signal processing pipelines or interactive queries. A subset of these languages is supported to enable static scheduling (e.g., only fixed loop iterations).

Listing 1 shows movement intent decoding with a Kalman filter, and Listing 2 shows a complex interactive query, both written in TrillDSP. The query detects seizures from signals in the last 5 s at all nodes, and sends the data 100 ms before/after detected seizures.
Listing 1: Movement Intent using a Kalman filter in TrillDSP.

$$var movements = stream \cdot window ( wsize = 50 ms ) \cdot sbp ( ) \cdot kf ( kf\_params ) \cdot call\_runtime ( )$$

Listing 2: Interactively querying seizure data.

$$var seizure\_data = stream \cdot Map ( // group by location s \text{=>} s \cdot select ( s \text{=>} s \cdot data ) , s \cdot locID ) \cdot window ( wsize = 4 ms ) \cdot select ( w \text{=>} w \cdot time >= - 5000 ) \cdot select ( w \text{=>} w \cdot seizure\_detect ( ) , w [ - 100 ms : 100 ms ] )$$

Programs are parsed into data flow directed acyclic graphs (DAGs). A configuration file maintains details of the components (the power consumption of PEs, radio data rates, etc.), overall constraints, and priorities (Section 3.5). The DAG and configuration file are used to formulate an ILP, which is solved with standard software.

The optimal schedule from the ILP solver contains the mapping of tasks to PEs and the network schedule. It is translated to assembly instructions that can be run on the per-node MCs. Translation occurs in two steps. From the ILP output, we first generate a C program using a library of predefined functions to configure the parameters of the PEs and their connections. Next, the program and the library are compiled to obtain RISC-V binaries. We also develop a lightweight runtime on the MC that listens to the external radio for data and code, and reconfigures PEs and pipelines.

### 4. DEPLOYING SCALO

FIG. 5 shows the seizure propagation pipeline on SCALO. The colors of the PEs are matched with the high level tasks from FIGS. 3A-C. In this pipeline, seizure detection uses FFT, Butterworth bandpass filters (BBF) and XCOR for feature extraction, followed by an SVM for classification. Signals are compared with DTW.

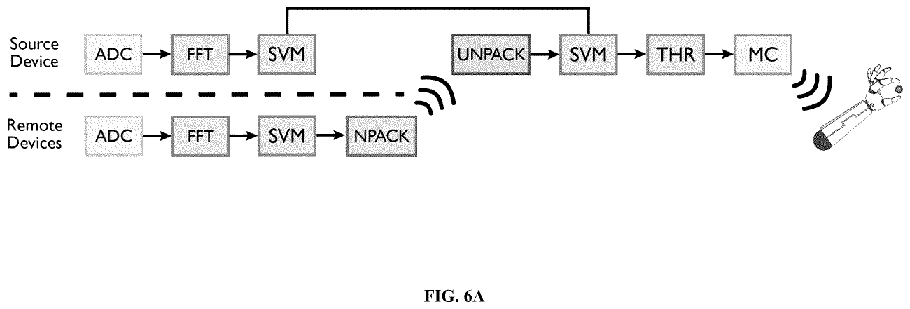

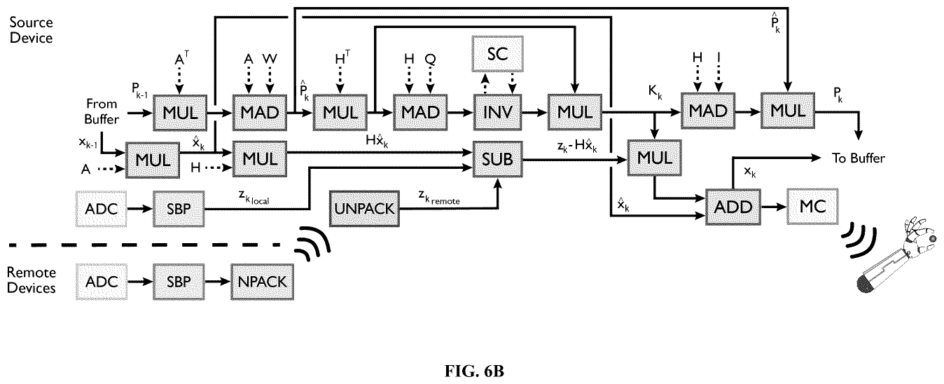

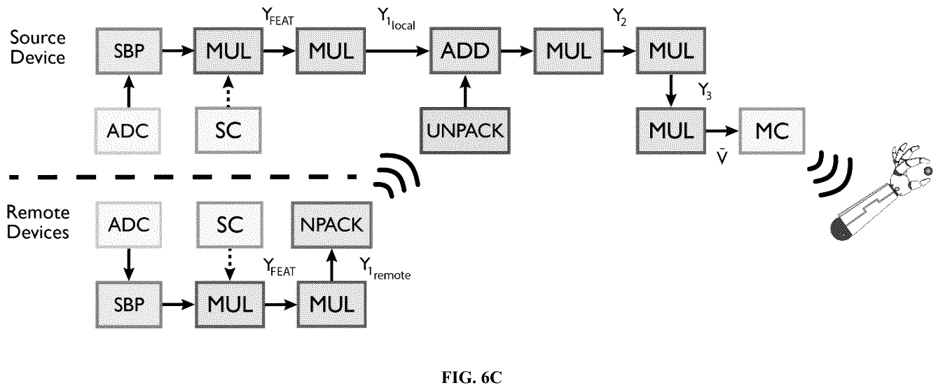

FIGS. 6A-C show the movement intent pipelines on SCALO. Algorithm A's implementation is derived from multiple sources. Algorithms B and C are implemented based on prior designs. In our implementation of Algorithm B, we do not change the Kalman filter parameters online as done in some variants although SCALO supports it.

In FIG. 6B, since the Kalman filter needs its output from the previous time step, we save this value to a buffer at the end of the pipeline. Additionally, the inversion operation (INV) needs to use the NVM because the matrix is too big to fit in the PE memory.

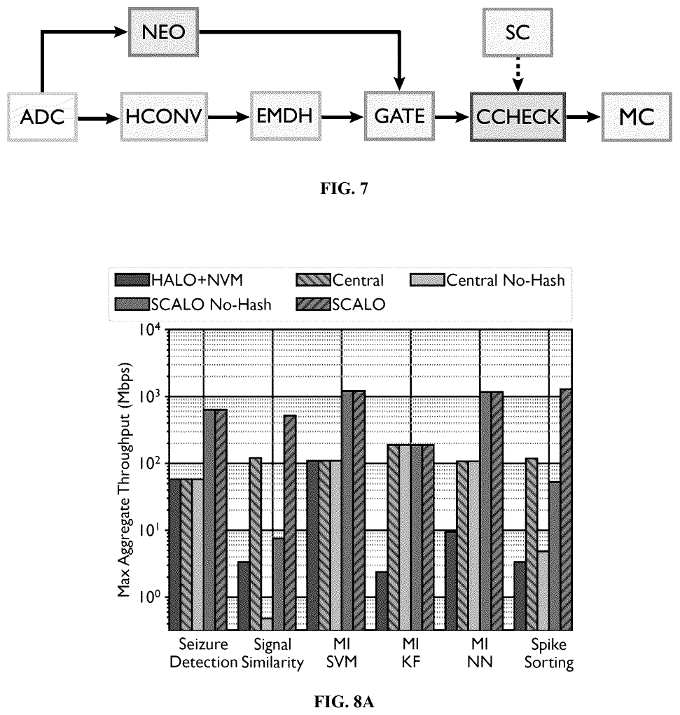

Last, FIG. 7 shows online spike sorting using EMD hashes and templates.

### 5. EXPERIMENTAL SETUP

Processing fabric: SCALO's PEs are designed and synthesized with Cadence tools at a commercial 28 nm fully-depleted silicon-on-insulator (FD-SOI) CMOS process. We use standard cell libraries from STMicroelectronic and foundry-supplied memory macros that are interpolated to 40° C., which is close to human body temperature. We design each PE for its highest frequency, and scale the power when using it at lower frequency. We run multi-corner, physically-aware synthesis, and use latency and power measurements from the worst variation corner. Table 1 shows these values. We confirm these values with partial tape-outs at 12 nm. In the table, blank entries indicate data-dependent latencies. The SC can take 0.03 or 0.04 ms depending on the NVM being available or busy.

TABLE 1
Latency and Power of the PEs.

| | Max | | | | | |
|---|---|---|---|---|---|---|
| Processing | Freq | Power (μW) | | | Latency | Area |
| Elements | (MHz) | Leakage (SRAM) | | Dyn/Elec | (ms) | (KGE) |
| ADD | 3 | 0.08 | (0.00) | 0.983 | 2 | 68 |
| AES | 5 | 53 | (0.00) | 0.61 | — | 55 |
| BBF | 6 | 66.00 | (19.88) | 0.35 | 4.00 | 23 |
| BMUL | 3 | 145 | (0.00) | 1.544 | 2 | 77 |
| CCHECK | 16.393 | 7.20 | (0.88) | 0.14 | 0.50 | 3 |
| CSEL | 0.1 | 4.00 | (0.00) | 6.00 | 0.04 | 2 |
| DCOMP | 16.393 | 7.20 | (0.00) | 0.14 | 0.50 | 3 |
| DTW | 50 | 167.93 | (48.50) | 26.94 | 0.003 | 72 |
| DWT | 3 | 4 | (0.00) | 0.02 | 4 | 2 |
| EMDH | 0.03 | 10.47 | (0.00) | 0.00 | 0.04 | 9 |
| FFT | 15.7 | 141.97 | (85.58) | 9.02 | 4.00 | 22 |
| GATE | 5 | 67.00 | (34.37) | 0.63 | 0.00 | 17 |
| HCOMP | 2.88 | 77.00 | (0.00) | 0.65 | 4.00 | 4 |
| HCONV | 3 | 89.89 | (0.00) | 0.80 | 1.50 | 8 |
| HFREQ | 2.88 | 61.98 | (0.00) | 0.52 | 4.00 | 6 |
| INV | 41 | 0.267 | (0.00) | 11.875 | 30 | 167 |
| LIC | 22.5 | 63 | (6.00) | 3.26 | — | 55 |
| LZ | 129 | 150 | (95.00) | 30.43 | — | 55 |
| MA | 92 | 194 | (67.00) | 32.76 | — | 55 |
| NEO | 3 | 12.00 | (0.00) | 0.03 | 4.00 | 5 |
| NGRAM | 0.2 | 15.69 | (9.07) | 0.08 | 1.50 | 10 |
| NPACK | 3 | 3.53 | (0.00) | 5.49 | 0.008 | 2 |
| RC | 90 | 29 | (0.00) | 7.95 | — | 55 |
| SBP | 3 | 12.00 | (0.00) | 0.03 | 0.03 | 6 |
| SC | 3.2 | 95.30 | (64.49) | 1.64 | 0.03-4 | 12 |
| SUB | 3 | 0.08 | (0.00) | 0.988 | 2 | 69 |
| SVM | 3 | 99.00 | (53.58) | 0.53 | 1.67 | 8 |
| THR | 16 | 2.00 | (0.00) | 0.11 | 0.06 | 1 |
| TOK | 6 | 5.57 | (0.00) | 0.14 | 0.001 | 3 |
| UNPACK | 3 | 3.53 | (0.00) | 5.49 | 0.008 | 2 |
| XCOR | 85 | 377.00 | (306.88) | 44.11 | 4.00 | 81 |

We assume that each node uses a standard 96-electrode array to sense neural activity, and has a configurable 16-bit ADC running at 30 KHz per electrode. The ADC dissipates 2.88 mW for 1 sample from all 96 electrodes. Each node also has a DAC for electrical stimulation, which can consume ≈0.6 mW of power.

Radio parameters: We use a radio that transmits/receives up to 10 m with external devices at 46 Mbps, 250 MHz, and consumes 9.2 mW. For intra-SCALO communication, we consider a state-of-the-art radio designed for safe implantation. We modify the radio, originally designed for asymmetric transmit/receive, for symmetric communication. The radio can transmit up to 20 cm (>90^{th} percentile head breadth). To estimate power and data rates, we use path-loss models with a path-loss parameter of 3.5 for transmission through the brain, skull, and skin, like prior studies. Our radio can transmit/receive 7 Mbps at 4.12 GHz and consumes 1.721 mW. We evaluate other radios in Section 7.

Non-volatile memory: We use NVMs with 4 KB page sizes and 1 MB block sizes. An NVM operation can read 8 bytes, write a page, or erase a block. We use NVSim to model NVM and set the SLC NAND erase time (1.5 ms), program time (350 us), and voltage (2.7 V) from industrial technical reports. We choose a low power transistor type, and use a temperature of 40° C. NVSim estimates a leakage power of 0.26 mW, dynamic energies of 918.809 nJ and 1374 nJ per page for reads and writes, respectively. We use these parameters to size our SC buffers to 24 KB.

Thermal and power limits: No brain region can be overheated beyond 1° C. The corresponding power cap depends on packaging, implantation depth, and, for multiple implants, the spacing among implants. Like prior work, we assume SCALO's implants are expected to be deployed as cuboidal strips or cylindrical capsules near the cortical surface, with the electrodes extending 1.5-2 mm into the brain gray matter. At this depth, no implant can dissipate more than 15 mW.

Earlier finite-element analyses of heat dissipation through brain tissues have shown that the temperature increase from an implant falls exponentially with distance, due to blood and cerebrospinal fluid flow. At 10 mm from an implant's edge, the temperature rise is ≈5% of the peak, and at 20 mm, the rise is only 2% with negligible thermal coupling between implants.

We use 20 mm as the default spacing in SCALO. Assuming uniform and optimal distribution of implants on a hemispherical brain surface of 86 mm radius, up to 60 SCALO implants can be run at 15 mW each, with negligible thermal coupling. Nonetheless, since node placement may vary by deployment, we report SCALO's performance when the nodes can consume only 12, 9, and 6 mW power, i.e., 60%, 40%, and 20% lower limits.

Electrophysiological data: We use publicly available electrophysiological data for our evaluation. For seizure detection and propagation, we use data from the Mayo Clinic of a patient (label “I001_P013”) with 76 electrodes implanted in the parietal and occipital lobes. This data was recorded for 4 days at 5 KHz, and is annotated with seizure instances. We upscaled the sampling frequency to 30 KHz, and split the dataset to emulate multiple implants.

We use overlapping 4 ms windows (120 samples) from the electrodes to detect seizures. For propagation, we compare a seizure-positive signal with the signals in the last 100 ms at all other nodes. When hashing, we use an 8-bit hash for a 4 ms signal.

We use three datasets to evaluate spike sorting. We use the Spikeforest dataset, with recordings collected from the CA1 region of a rat hippocampus using tetrode electrodes at 30 KHz sampling frequency. The dataset contains spikes from 10 neurons, with 65,000 spikes from 4 channels that were manually sorted. We also use the Kilosort dataset, which has 35,000 spikes from 30 neurons collected with a neuropixel probe with 384 channels. Finally, we use the MEArec dataset, which contains 4,544 spikes from 20 neurons, generated using a neuron cell simulation model.

Alternative system architectures: Table 2 shows the systems that we compare SCALO against. SCALO No-Hash uses the SCALO architecture but without hashes. The power saved by removing the hash PEs is allocated to the remaining tasks optimally. Central No-Hash uses a single processor without hashes like most existing BCIs. The processor is connected to the multiple sensors using wires. Central is another single-processor design, but uses hashes like SCALO. Finally, we have HALO+NVM, which uses a single HALO processor, augmented with an NVM to support our applications. Since this design does not have our new PEs, it uses the RISC-V processor for tasks like hashing.

TABLE 2
Alternative BCI architectures.

| Design | Architecture | Comparison | Communication |
|---|---|---|---|
| SCALO (proposed) | Distributed | Hash, Signal | Wireless |
| SCALO No-Hash | Distributed | Signal | Wireless |
| Central No-Hash | Centralized | Signal | Wired |
| Central | Centralized | Hash, Signal | Wired |
| HALO + NVM | Centralized | Hash, Signal | Wired |

We do not consider (1) wired distributed designs because it is impractical to have all-to-all wires on the brain surface, (2) wireless centralized designs as they have lesser compute available than the wired ones, and (3) designs without storage since all our applications need it. We map the applications onto all systems using the ILP, ensuring that each implant consumes<15 mW.

### 6. EVALUATION

### 6.1 Comparing BCI Architectures

We compare BCI architectures using their “maximum aggregate throughput” per application. This value is the throughput achieved (over all nodes) for an application when it is the only one running on SCALO. Aggregate throughput is calculated by increasing the number of electrode signals (and ADCs) that the node can process until the available power is fully utilized, or response time is violated. We consider a total of 11 implanted sites, which has the highest seizure propagation throughput for SCALO and SCALO No-Hash (Section 6.3). We later vary the number of nodes.

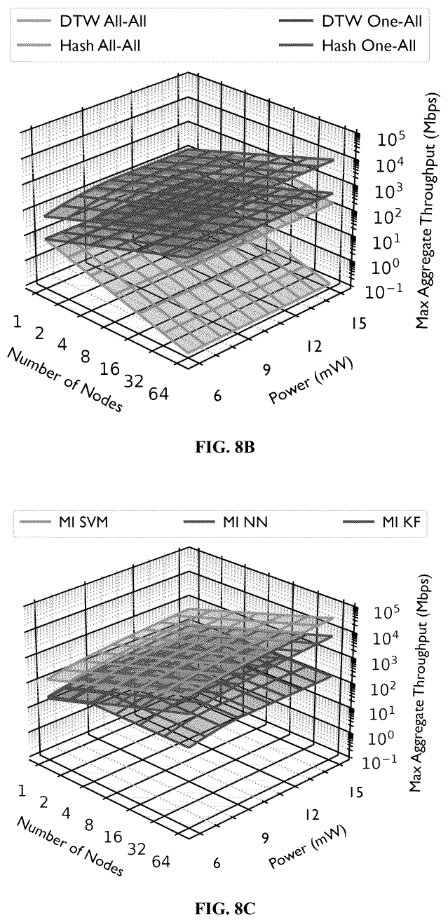

FIG. 8A shows performance results. We separate seizure detection and signal similarity in the seizure propagation application, since the former is local while the latter is distributed. Among the centralized designs, HALO+NVM does not have SCALO's new PEs but has the same performance as Central and Central No-Hash for seizure detection and SVM-based movement intent (MI SVM). This is because the PEs in HALO+NVM are sufficient for these tasks. On the other hand, HALO+NVMis 10-100× worse than Central for the remaining tasks because they are run on a slow microcontroller. For the spike sorting application, despite using hashing, HALO+NVM has a 40% lower throughput than Central No-Hash because checking for hash collisions on the microcontroller is slower than running an exact comparison on a PE in Central No-Hash. This performance gap highlights the need for hardware acceleration.

Central No-Hash has 250× and 24.5× lower throughput than Central for signal similarity and spike sorting respectively. These tasks benefit from hashes while Central No-Hash does not support hashing. The impact of not hashing is much more pronounced for signal similarity because this task involves inter-implant communication. Without hashes, the number of signals that can be communicated and compared under the power limit is low.

Central performs best among uniprocessor designs. However, the processor is the bottleneck for multi-site interfacing, and Central has 10× lower throughput than SCALO for all applications. One exception is the movement intent with Kalman filter (MI KF) application. In this case, SCALO also centralizes the computations (Section 3.1), resulting in a similar throughput for the two designs.

With SCALO No-Hash, overall processing capability scales with number of implants, as seen by throughput for seizure detection and MI SVM. However, SCALO No-Hash does not use hashing and performs worse than Central for signal similarity and spike sorting.

Finally, SCALO has the highest throughput for all applications. SCALO's LSH features enables scaling with more implants. Compared to HALO+NVM, which is the state-of-the-art prior work, SCALO's processing rates are 10× higher for seizure detection and MI KF, and are up to 385× higher for the remaining applications.

### 6.2 Performance Scalability

We evaluate the performance (maximum aggregate throughput) of SCALO for our applications with various node counts and per-node power limits. Among the applications, the seizure detection task and spike sorting are fully local to each node. Among these, seizure detection has more complex operations than spike sorting. The throughput of seizure detection at 15 mW is 79 Mbps and falls quadratically to 46 Mbps at 6 mW. Spike sorting has a throughput of 118 Mbps at 15 mW, which decreases linearly to 38.4 Mbps at 6 mW.

For the remaining applications, which are distributed, FIGS. 8B and 8C show their performance scaling with varying node counts and power limits. FIG. 8B shows the performance of hash and exact (DTW) signal similarity methods separately, under two communication patterns each. The first is all-to-all, which is the worst case communication pattern that occurs when brain-wide correlations must be identified, e.g., when there is a seizure at all nodes. The other is one-to-all communication, which occurs when only a single node detects a seizure and must broadcast its data.

DTW All-All has the least throughput because only 16 electrode signals can be transmitted in this mode. The reason is that the intra-SCALO radio can only transmit ≈7 Mbps, while new electrode samples are obtained at 46 Mbps from the ADC. Increasing the number of nodes decreases the throughput further because each node must serially access the TDMA network. Being communication-limited, DTW All-All is unaffected by lowering power limits even up to 6 mW. The DTW PE only needs 4 mW to process data at the available radio transmission rate, and its throughput scales linearly with power only below 4 mW.

DTW One-All scales better as its communication cost is fixed. However, a one-to-all comparison is insufficient for general BCI applications. DTW One-All is also communication-limited like DTW All-All and remains unaffected by lower power up to 4 mW.

The throughput of Hash All-All is 10× higher than that of DTW One-All for node counts ≤6. Relative to DTW All-All, the performance advantage is even higher. Hash All-All throughput linearly increases up to 547 Mbps (for 6 nodes with 190 electrode signals), after which it begins to decrease. When the number of nodes is small, few TDMA slots are required to exchange all hashes, allowing throughput to linearly increase with node count. When node count increases beyond a limit (i.e. 6), it takes longer to communicate all hashes and overall throughput reduces. Hash One-All has a 10× higher throughput than even Hash All-All, and exhibits linear scaling since the communication cost is fixed.

Hash processing is not communication-limited, as the transmitted is small (1 B per electrode versus 256 B for DTW). Throughput falls linearly when the power limit is lowered. Keeping number of nodes equal, for Hash All-All, peak throughput reduces from 547 Mbps at 15 mW to 135.35 Mbps at 6 mW, while for Hash One-All, it reduces from 6,851 Mbps at 15 mW to 1,444 Mbps at 6 mW.

FIG. 8C shows the performance of the movement intent applications. These use an all-to-one communication pattern: MI KF sends the features from all nodes, while MI SVM and MI NN send partial classifier outputs to one node. MI SVM transmits only 4 B per node even if the number of electrodes per node goes beyond 96, because it only needs to send the partial classifier output and not electrode data. Additionally, for a given power limit, MI SVM can process 3% more electrodes than hash generation because the SVM PE consumes 3% lower power than the hash PEs. Therefore, MI SVM has the highest throughput than even Hash One-All, which also scales linearly with node size.

MI NN, like MI SVM, has a fixed data transmission size per node, but the size of this data is higher (1024 B). Therefore, it has a lower throughput than MI SVM but has the same scaling trend. Both are power limited and see a linear decrease in throughput with power.

In contrast to the other MI applications, MI KF transmits much higher data at 4 B per electrode, since it transmits only features for centralized processing. Furthermore, the inversion step in MI KF at the receiver has a high usage of the NVM. Therefore, MI KF's throughput scales linearly only up to 4 nodes, where the NVM bandwidth saturates and the application cannot process any more electrodes in the given response time. Therefore, with higher number of nodes, the number of electrodes that can be processed per node decreases, and overall throughput remains the same.

MI KF is limited only by NVM bandwidth above 8.5 mW power, and does not see any throughput reduction until the power limit reaches this value. Below this, throughput falls off quadratically.

### 6.3 Application Performance

We measure application-level performance via throughput for seizure propagation, number of intents per second for the movement applications, and the spikes sorted, for various node counts.

Seizure propagation has multiple inter-related tasks since seizure detection can run concurrently with hash or DTW comparison, and there is a choice between sending more hashes or signals in the given response time. Hence, it is necessary to specify priorities for these tasks to determine the application performance. Recall that the ILP maximizes the priority-weighted sum of the signals processed in the tasks. Although the ultimate choice of weights is determined by a clinician, we evaluate three sets of weights.

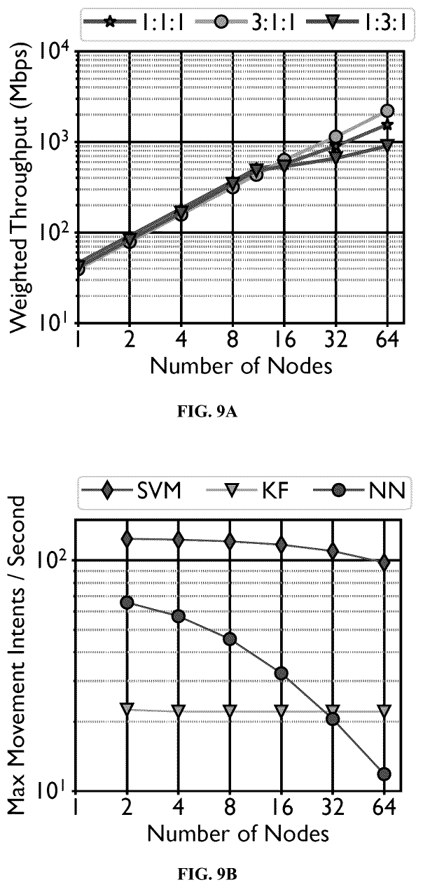

FIG. 9A shows the maximum weighted aggregate throughput for seizure propagation with different weight choices (in the format; seizure detection:hash comparison:DTW comparison). With equal priority for all tasks, throughput increases linearly up to 506 Mbps, achieved at 11 nodes. The highest throughput per node is achieved at this node count. Beyond this value, overall throughput increases sublinearly due to communication costs. Other weight choices have different throughput and optimal node counts.

Conventional movement intent (MI) applications use a fixed time interval (e.g., 50 ms) to detect one intent. This limits the number of intents detected (i.e., 20 per second). SCALO decodes movements much faster than this interval.

FIG. 9B shows the maximum number of intents detected per second on SCALO. This metric only accounts for intent detection, without the variable response latency of the prosthetic. SCALO significantly outperforms conventional MI SVM and MI NN, which offer only 20 intents per second and for a few electrodes (not shown in the figure). For MI KF, which is the most complex MI application, SCALO also supports 20 intents per second but can process up to a total of 384 electrodes, which is up to 4 nodes for a 96-electrode node. For higher node count, SCALO can still retain its performance but the electrodes processed per node decrease.

Finally, SCALO sorts up to 12,250 spikes per second per node by using hashes to match spikes with preset templates on the NVM. For reference, leading off-device exact matching algorithms sort up to ≈15,000 spikes per second but use multicore CPUs or GPUs. The sorting accuracy of SCALO is within 5% of that achieved by exact template matching, which is 82%, 91%, and 73%, respectively for the SpikeForest, MEArec, and Kilosort datasets.

### 6.4 Interactive Queries

We consider three common queries for data ranging from the past 110 ms (≈7 MB over all nodes) to the past 1 s (≈60 MB). They are: Q1, which returns all signals detected as a seizure; Q2, which returns all signals that match a given template using a hash; and Q3, which returns all data in the time range. For Q1 and Q2, we set the fraction of data that tests positive for their condition at 5%, 50%, and 100%.

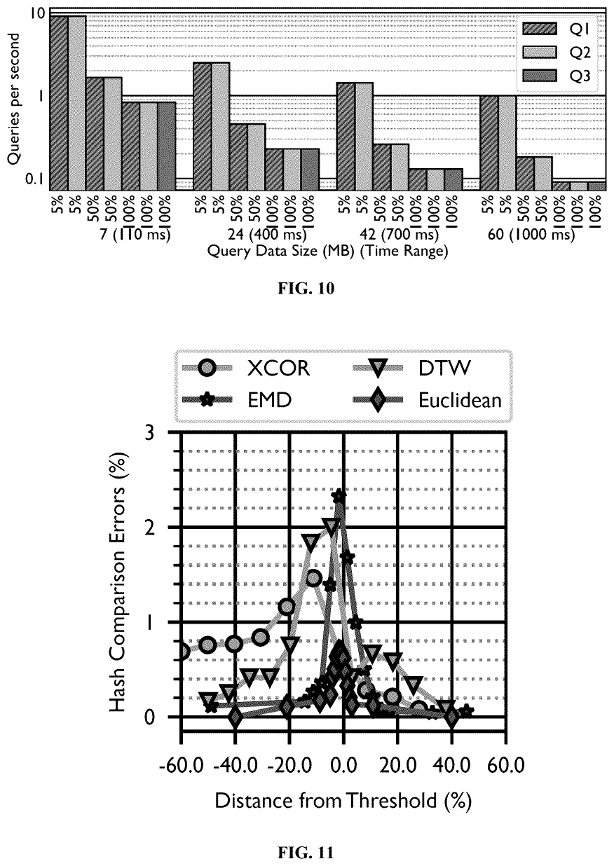

FIG. 10 shows SCALO's throughput with 11 nodes for our queries. SCALO supports up to 9 queries per second (QPS) for Q1 and Q2 over the last 110 ms data for 5% matched data, which is the common range of data queried over. If Q2 is run with DTW instead of hashes, the QPS is 8, which is only slightly lower, but the power consumption increases to 15 mW instead of the 3.57 mW consumed with hash-based matching. DTW-based matching is unsuitable when interactively querying in response to a seizure. Q3 takes 1.21 s, yielding a throughput of ≈0.8. The power-hungry external radio becomes the bottleneck for interactive querying. Query latency increases linearly with more search data because of radio latency. Still, SCALO can processes 1 QPS for Q1 and Q2 for the past 1 s data (≈60 MB) with 5% matched data, making it usable in real time.

### 6.5 Hash Encoding Accuracy

We measure the accuracy of hash-based signal comparison relative to exact comparison for various measures. For each measure, we set a similarity threshold. If the measure for a given pair of signals is above the threshold, they are considered similar. We then configure our hash generation functions for this threshold, and check for the same outcome using hashes, i.e., only similar signals should generate the same hash. FIG. 11 shows the percentage of errors between hash and signal comparison as a function of the signals' distance from the threshold. The total errors, represented as area under the curve, are few at <8.5%. Most hash errors occur close to the threshold, where even exact comparison is of low confidence in identifying a match, and errors taper off quickly with distance from the threshold. Note that we bias the hashes towards false positives (left of the threshold), since they can be resolved by an exact comparison.

### 6.6 Impact of Network Errors

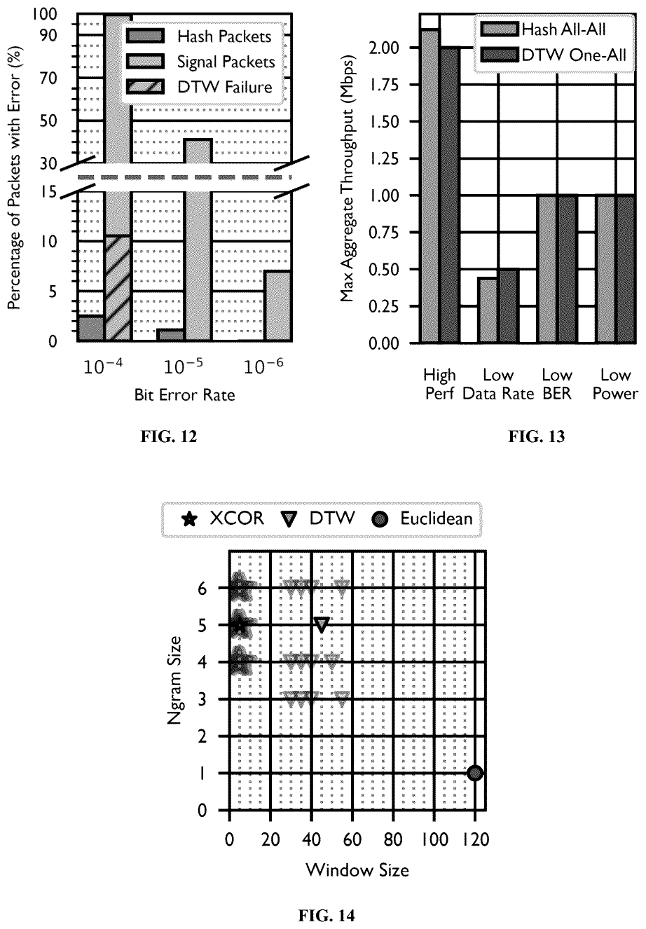

The intra-SCALO network protocol drops packets carrying hashes when there is a checksum error, but allows signal packets to flow into PEs since signal similarity measures are naturally resilient to errors. We simulate bit-error ratios (BERs) with uniformly-random bit flips in packet headers/data. FIG. 12 shows the fraction of hash/signal packets with an error at different BERs, and the fraction of erroneous signal packets that flipped the similarity measure (DTW). The BER is <10⁻⁵ for the radio we use.

FIG. 12 shows that signals and hashes suffer errors as BER increases, but signals are more susceptible since they are longer. However, even though many signal packets suffer errors, they have no impact on the final signal similarity outcome since the measures are naturally resilient. For our design (BER<10⁻⁵), <1% of hash packets have errors and there is no DTW failure.

### 6.7 Error Impact on Applications

Hash errors, caused either due to incorrect encoding or network faults, can affect application performance. However, signals in the brain are spatially and temporally correlated, providing some resiliency to such errors. We use the time-sensitive seizure propagation application to study the impact of hash errors. In this application, a false negative or a hash packet error can cause seizure propagation confirmation to be delayed.

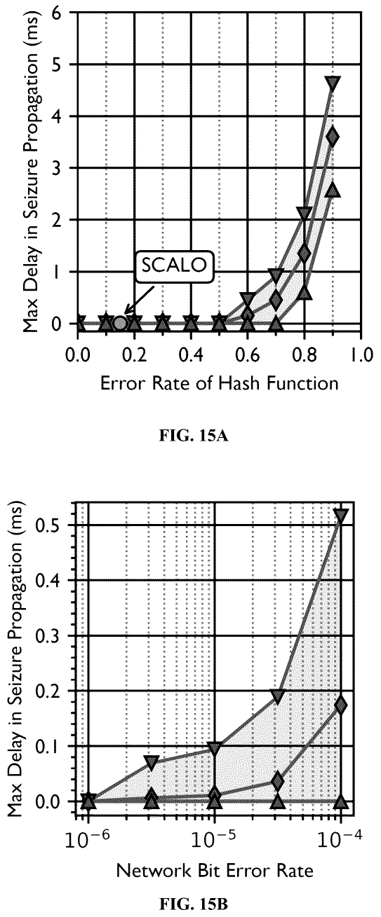

FIGS. 15A-B show the maximum delay in seizure propagation for each type of error when there is a correlated seizure in two brain regions, showing the 100% intervals after 1000 repetitions. FIG. 15A shows that hash encoding errors (which in this case are false negatives because there is an ongoing correlated seizure) do not cause any noticeable impact until the error rate is around 50%. The reason for this resiliency is that when a seizure occurs, it is captured by multiple electrodes. It is highly unlikely that all such signals are incorrectly encoded to completely miss the correlation at this time step. For reference, we observe only 12.5% of false negatives in SCALO. Furthermore, a seizure lasts for a few seconds, meaning that another round of correlation checking can occur at the next time step even if it is missed in the current time step.

FIG. 15B shows the application-level delay with network BER. Recall that all hashes from a node can be sent in one packet. There-fore, a network error results in the loss of the hashes from all electrodes at a node, and correlation can resume only at the next time step. Consequently, network errors are more harmful than encoding errors. However, these errors are also much less likely to occur (note that the Y axis of FIG. 15B is different from FIG. 15A), and the worst delay even at a BER of 10⁻⁴ is 0.5 ms. For reference, the radio we use has a BER of 10⁻⁵.

### 7. DESIGN SPACE EXPLORATION

Hash Parameter selection: FIG. 14 shows the best parameters for LSH (window size and n-gram size-Section 2.4) to approximate different signal measures. We also show parameters (with lighter colors in the figure) that are within 90% of the true positive rate achieved by the corresponding best configuration. This flexibility enables reusing the same hash (and PEs) for different measures.

Radio parameters: There are many radio designs for safe implantation with various trade-offs between the data rate, power, and BER. We evaluate the performance of hash (All-All) and DTW (One-All) with four such radios listed in Table 3. For all radios, we maintain a transmission distance of 20 cm and scale the remaining parameters appropriately for this distance. Low Power is our default radio.

TABLE 3
Alternative radio designs. Our choice is Low Power

| Name | BER | Data rate (Mbps) | Power (mW) |
|---|---|---|---|
| Low Power | 10⁻⁵ | 7 | 1.71 |
| High Perf | 10⁻⁶ | 14 | 6.85 |
| Low BER | 10⁻⁶ | 7 | 3.4 |
| Low Data | 10⁻⁵ | 3.5 | 0.855 |
| Rate | | | |

FIG. 13 shows the throughput of the applications with the different radios, normalized with that of our default choice (Low Power). The High Perfradio doubles the throughput of both applications because they are communication sensitive. However, the radio power becomes 4×, occupying nearly half the available 15 mW budget. The Low BER radio has the same performance as our default, but has 2× the power. This trade-off is not advantageous since our BER is already low (10-5). Lastly, the Low Data Rate radio results in a 50% lower performance for the applications, which is unacceptable for our response time targets.

### 8. RELATED WORK

Single-site BCIs: Commercial and research BCIs have focused largely on single-site monitoring and stimulation, and have no support for distributed systems, making them unsuitable for the applications that we target. Most implantable BCIs offer little to no storage and stream data out continuously instead. NeuroChip is an exception but is wired to an external case with a 128 GB SD card that is physically extracted for offline data analysis.

Distributed implants: A growing interest in distributed analyses of the brain has motivated the design of multi-site BCIs. These BCIs, however, lack on-board processing and stream data to a separate computer, or a chest or scalp mounted processing hub. Unfortunately, such centralization restricts the response time and throughput of the BCI, limiting its utility for distributed applications.

Implantation architecture: SCALO presents just one example of a distributed BCI. Alternative designs could include hubs that are chest-implanted, or scalp mounted. Hubs can serve as wired sources of power for the implants, while the hub itself could be powered by removable or wirelessly charged batteries (it is less risky to wirelessly charge a chest-implanted or externally mounted device). The hub may also act as the sole processor in the system, using the distributed implants only as sensors. Yet another approach is to use wearable hubs. The SCALO architecture can be adapted to suit these various scenarios, although one or more functionalities may not be applicable.

Accelerators for BCI applications: Recent work has designed specialized hardware accelerators for spike sorting using template matching, and DNN accelerators for classification using unary networks. These designs are promising, particularly for applications permitting higher power consumption.

### 9. CONCLUSION

SCALO enables BCI interfacing with multiple brain regions and provides, for the first time, on-device computation for important BCI applications. SCALO offers two orders of magnitude higher task throughput, and provides real-time support for interactive querying with up to 9 QPS over 7 MB data or 1 QPS over 60 MB data. SCALO's design principles—i.e., its modular PE architecture, fast-but-approximate hash-based approach to signal similarity, support for low-power and efficiently-indexed non-volatile storage, and a centralized planner that produces near-optimal mapping of task schedules to devices—can be instrumental to success in other power-constrained environments like IoT (internet of things) as well.

### 10. ARTIFACT APPENDIX

### Abstract

There are 4 artifacts for this Example: hash function library to reproduce FIG. 11, a python program to reproduce FIG. 12, a basic query processor to generate ILP programs to recreate FIGS. 8A-9B. The artifact also contains a Dockerfile to install all requirements and run all tests to provide a push button solution.

### Artifact Check-List (Meta-Information)

Programs: glpsol, python3, Docker, NVSim
Compilation: Artifact includes scripts to compile NVSim from source
Data set: Artifact includes data collected from patient with label I001_P013 downloaded from ieeg.org
Run-time environment: Experiments are run on a Docker container running Ubuntu 22.04, Linux 5.19
Hardware: A Linux System with Intel X86-64 CPU, 8 GB RAM.
Metrics: 1) Application throughput calculated using ILP 2) Hash function error rates 3) Packet loss due to Bit Error Rate
Output: Output generates plots in the paper, as described in sections further
Experiments: Experiments measure hash error rates, and the packet loss due to bit errors
How much disk space required (approximately)?: 15 GB
How much time is needed to prepare workflow (approximately)?: 10-15 minutes
How much time is needed to complete experiments (approximately)?: 30-45 minutes
Publicly available?: Yes, 10.5281/zenodo.7787128
Code licenses (if publicly available)?: CC4
Archived?: 10.5281/zenodo.7787128

### Processing Elements

Table 4 lists all our PEs.

TABLE 4
Processing Element Names

| Name | Function |
|---|---|
| ADD | Matrix Adder |
| AES | AES Encryption |
| BBF | Butterworth Bandpass Filter |
| BMUL | Block Multiplier |
| CCHECK | Collision Check |
| CSEL | Channel Selection |
| DCOMP | Decompression |
| DTW | Dynamic Time Warping |
| DWT | Discrete Wavelet Transform |
| EMDH | Earth-Mover's Distance Hash |
| FFT | Fast Fourier Transform |
| GATE | Gate Module to buffer data |
| HCOMP | Hash Compression |
| HCONV | Hash Convolution Operation |
| HFREQ | Hash Frequency |
| INV | Matrix Inverter |
| LIC | Linear Integer Coding |
| LZ | Lempel Ziv |
| MA | Markov Chain |
| NEO | Non-linear Energy Operator |
| NGRAM | Hash Ngram Generation |
| NPACK | Network Packing |
| RC | Range Coding |
| SBP | Spike Band Power |
| SC | Storage Controller |
| SUB | Matrix Subtractor |
| SVM | Support Vector Machine |
| THR | Threshold |
| TOK | Tokenizer |
| UNPACK | Network Unpacking |
| XCOR | Pearson's Cross Correlation |

### 10.1 Description

10.1.1 How to access. You can access the artifact at https://zenodo. org/record/7787128
10.1.2 Hardware dependencies. A Linux machine with Docker in-stalled, about 8 GB RAM, and 15 GB free disk space. The instructions to run the experiments are specific to Linux on X86, but the artifacts may work in other environments.
10.1.3 Software dependencies. DockerOur experiments can be run quickly using scripts on Docker, though we also specify how to run the experiments in python directly.

SoftwarePython3, python libraries (matplotlib, numpy, scipy, statsmodel, python-dtw), glpsol (from glpk-ultils package)

10.1.4 Data sets. Data collected from patient with label I001_P013 downloaded from ieeg.org. This data set contains EEG signals collected from 76 electrodes implanted in the parietal and occipital lobes, recorded at 5 KHz. The dataset was upscaled to about 30 KHz an split to multiple filles to simulate multiple BCI devices.

### 10.2 Description

For a push button solution, install Docker using your Linux Distribution's software installer. You can then create a container that immediately runs all experiments. Run the following command after extracting the artifact in the folder containing the Dockerfile—
$: docker build -t hull-archive.

This will start building an Ubuntu container, installing all dependencies, then automatically running the experiments using the scripts we provide.

Alternatively, you may run the experiments locally. The experiments depend on python3 and certain python3 libraries. These libraries can be installed by first installing python3 and pip3 using your distribution's installer. Following that, you can run—
$: pip3 install -r work/requirements.txt
   inside the root directory of the artifact.

In addition to python, some experiments also use the GNU LP solver, glpsol. This can be installed using your distribution's installer (eg by installing glpk-utils on Ubuntu).

### 10.3 Experiment Workflow

If you set up Docker, the experiment would run automatically. After the docker container is setup, you can copy the results on to the host machine by running the following commands in separate terminals.
$docker run -name artifact -it hull-archive
$docker cp artifact:/work/path/in/host/to/store/

You can access all results (pdf files) in the respective directories and view them by running—
$: ls work/*/*.pdf.

If you have set up a local install, you can run all experiments by navigating to the work folder in artifact, then running—
$sh script.sh

Which will start running experiments one by one. This is expected to take 20-30 minutes. Once done, you can access the results in the same way as mentioned above.

### 10.4 Evaluation and Expected Results

Hash Error Rates: This experiment evaluates the error rates of the hash functions we describe in the paper, It exists inside work/hash directory and is run using—
$: python3 hash_err_rates.py

It produces the hash_hist.pdf file in the same directory, and should look similar to FIG. 10. There may be slight differences due to randomization but such errors should be minimal (≈1-2% absolute error)

Network Bit Error Rates: This experiment evaluates the impact of bit errors on the end application accuracy, and recreates FIG. 12. It exists inside work/ber directory, and is run using—
$ python3 network ber.py

Task throughput This experiment recreates FIGS. 8A-C. The figures can be generated directly by running fig7a.sh, fig7b.sh, and fig7c.sh inside the work/ilp directory. Each script setups up helper python scripts to use hardware information and application query to generate an ILP program. This ILP program is then solved for an optimal solution using glpsol, and then plotted using more helper python scripts. The hardware information of the device is stored in HALO.json for convenient access. Please refer to Table 4 to understand function of each PE. Examples of queries are stored in txt files in the same directory, e.g. seizure_detection.txt stores the query to run a seizure detection application. The directory also contains a readme.md file that explains the grammar of the queries. You may also read the shell script files for examples on running a query.
describe_device.py is a helper python script to generate json files for hardware
create_ilp.py takes in the hardware information json file, a query file, and a target number of devices to generate an ILP program to find an optimal schedule for it on the SCALO system.

Application Level Throughput: This experiment recreates FIGS. 9A-B. While these experiments may be run on the ILP as well, they take a long time to run due to the larger number of variables and devices in the ILP program. To obtain results faster, we have taken reduced linear equations that resulted from a prior solution to quickly plot solutions for larger problems. This can be generated by running $: python3 work/lineqn/seizure_Plus_hash.py. The equations, and their weights are stored and explained inutils.py.

NVSim: This experiment shows the configuration for the NVM used in SCALO. This is stored in work/NVSim/HULL.cfg. You can then run $: ./nvsim HULL.cfg to view the energy, and bandwidth numbers estimated by NVSim. Particularly, the tool estimates leak-age power to be 0.26 mW, and dynamic energies of 918.809 nJ and 1374 nJ per page for reads and writes, respectively.

### 10.5 Experiment Customization

Our scripts are set up to allow easy extension, customization, and experimentation. The hash error program is set up to be run on different input files with a small modification, along with code to allow fast exploration of all parameters of the hash. The ILP is set up to allow queries of different kinds with a readme explaining writing custom queries.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for communicating data between nodes in a computer system, the method comprising:
generating a hash based upon a data set;
communicating the hash to one or more nodes;
comparing the communicated hash to stored hashes at the one or more nodes; and
communicating the data set when matching hashes are detected.

2. The method of claim 1, wherein detecting matching hashes comprises determining that the underlying data sets are likely to be correlated.

3. The method of claim 2, wherein determining that the underlying data sets are likely to be correlated includes applying a suitable similarity measure.

4. The method of claim 3, wherein the similarity measure includes Euclidean distance, cross-correlation (XCOR), dynamic time warping (DTW), Earth Mover's Distance (EMD), or a combination thereof.

5. The method of claim 1, wherein at least one of the nodes locally generates and stores one or more of the hashes.

6. The method of claim 5, wherein the at least one node locally generates and stores the one or more hashed based upon locally collected data.

7. The method of claim 6, wherein the at least one node includes an originating node and a receiving node.

8. The method of claim 7, wherein the originating node communicates the locally generated hash to the receiving node.

9. The method of claim 8, wherein the receiving node compares the locally generated hash communicated from the originating node to a stored hash locally generated at the receiving node.

10. The method of claim 9, wherein the receiving node responds to the originating node when there is a match between the locally generated hash communicated from the originating node and the stored hash locally generated at the receiving node.

11. The method of claim 10, wherein the originating node communicates the locally collected data only when the receiving node responds.

12. A distributed system of computer architectures comprising:
two or more processing elements;
wherein the processing elements are configured to communicate data according to the method of claim 1.

13. The system of claim 12, wherein the processing elements are connected through a wireless network, a wired network, wires on a chip, or a combination thereof.

14. The system of claim 12, wherein the processing elements include application-specific integrated circuits (ASICs).

15. The system of claim 14, wherein the ASICs are configured to realize a plurality of hashes in low power.

16. The system of claim 12, wherein each of the processing elements is built in an independent clock domain.

17. The system of claim 12, wherein the distributed system comprises a brain-computer interface (BCI) architecture for multi-site brain interfacing in real time.

18. The system of claim 12, wherein the system is resource-constrained.
